(19) United States
(12) Patent Application Publication
(10) Pub. No.: US 2022/0167839 A1
(43) Pub. Date: Jun. 2, 2022

(54) AN ENDOSCOPE HAVING A LIGHT EMITTING MEANS AND IMAGE PICKUP DEVICE PROVIDED ON THE DISTAL END PORTION

(71) Applicant: HOYA CORPORATION, Tokyo (JP)

(72) Inventors: Anh Minh DO, Friedberg (DE); Tilman SCHRÖTER, Friedberg (DE)

(73) Assignee: HOYA CORPORATION, Tokyo (JP)

(21) Appl. No.: 17/434,610

(22) PCT Filed: Mar. 3, 2020
(86) PCT No.: PCT/IB2020/051770

§ 371 Date: Aug. 27, 2021

(30) Foreign Application Priority Data
Mar. 6, 2019 (DE) 10 2019 105 671.3

(51) Int. Cl. A61B 1/05; A61B 1/06; A61B 1/273; A61B 1/00

(52) U.S. Cl. CPC A61B 1/05; A61B 1/00177; A61B 1/2736; A61B 1/06

(57) ABSTRACT

The invention relates to an endoscope having a distal end portion, at which a light emitting means and an image pickup device are arranged, wherein, at the distal end portion, a light incidence region is defined at the outer circumference of the distal end portion. The longitudinal axis of the image pickup device is oriented towards the light incidence region at the outer circumference of the distal end portion.

The present invention relates to an endoscope having a distal end portion, on which a light emitting means and an image pickup device are provided.

An endoscope is an instrument for examining the interior of living organisms, but also for examining technical cavities. An important part of an endoscope is the distal end portion, on which a light emitting means and an image pickup device are provided. The distal end portion is inserted into the interior of the organism or of the technical cavity. The light emitting means illuminates the inside of an organism or of a technical cavity to allow capturing of an image of the surrounding area. The image of this area can be forwarded to evaluation or can represent the basis of specific decisions regarding the interior of the organism or of the technical cavity.

After the examination, the endoscope can be removed from the organism or the technical cavity. After removing the endoscope and, in particular, the distal end portion, it will be cleaned and sterilized.

After cleaning and sterilization, in particular the distal end portion shall be residue-free and free from germs.

The document DE 10 2015 113 016 A1 discloses an endoscope head. On the distal side of the endoscope head, a pivot element operable from the proximal side is provided. On the distal end portion of the working channel, a pivoting Albarran lever, which is rotatably supported on a cap is provided. The cap is removable from the endoscope head. At the distal end of the endoscope head, a camera, an illumination means and the Albarran lever including a control mechanism are provided. The camera is provided on the one side and the control mechanism of the Albarran lever on the other side of the distal end. The Albarran lever is pushed into the space in the center. When the cap is removed the space in the center is exposed. The cap and the Albarran lever can be designed as a single-use assembly. The space in the center is cleaned and sterilized. The optical assembly of the endoscope head includes a camera disposed parallel to the axis of the endoscope head. Between the camera and a lens, a prism is provided which deflects light rays incident on the lens to the camera. Such an optical assembly is complex and expensive.

It is the object of the present invention to create an endoscope, where the assembly of the distal end portion is designed in a simpler and more cost-effective manner.

This object is achieved by an endoscope having the features of claim 1. Advantageous further developments are contained in the dependent claims.

The invention relates to an endoscope comprising a distal end portion, on which a light emitting means and an image pickup device are provided. On the distal end portion, a light incidence region is defined at the outer circumference of the distal end portion. The longitudinal axis of the image pickup device is oriented towards the light incidence region at the outer circumference of the distal end portion.

Such an optical assembly spares a prism. The distal end portion becomes simpler and cheaper since the relatively expensive prism is not required.

With such an endoscope, the light emitting means and the image pickup device can be located closely together and compactly on a portion of the distal end portion. The portion of the distal end portion on which the light emitting means and the image pickup device are located can be e.g. a side portion of the distal end portion. In the longitudinal direction of the endoscope, a space can be provided adjacent to the said side portion, in which a pivoting Albarran lever is located. Since, on the side portion of the distal end portion, the light emitting means and the image pickup device are accommodated compactly and closely together space becomes available on the same side portion of the distal end portion, which can be used for other purposes. The said space which has become available can be used e.g. for the driving mechanism of the Albarran lever. If the driving mechanism of the Albarran lever is provided on the same side portion of the distal end portion as the light emitting means and the image pickup device the overall distal end portion can be designed compactly and with an external shape which is easy to clean. The space that has become available on the side portion of the distal end portion can also be used for example for a flushing nozzle etcetera. Alternatively, the space that has become available on the side portion of the distal end portion can be used for an air nozzle for expanding the tissue to allow a better view behind folds of the intestine. This allows to gain images of areas which are otherwise inaccessible.

Thus, the space which has become available can be used in diverse ways.

With the present endoscope, the longitudinal axis of the image pickup device can extend perpendicularly to the light incidence region at the outer circumference of the distal end portion. Thereby, the image pickup device requires very little space in the distal end portion. In the longitudinal extension direction of the distal end portion, the space requirement of the image pickup device can be reduced to a minimum.

The distal end portion can have a distal extension extending from the distal end portion in the distal direction. The distal end portion can include an Albarran lever extending from the distal end portion in the distal direction. On the distal extension, the image pickup device can be provided.

The Albarran lever can be supported in a hinged manner on the distal extension. The distal extension can, in this case, include a coupling element by which the Albarran lever can be connected. Hence, an endoscope having a distal end portion can be created, where the Albarran lever can be decoupled, i.e. removed, from the distal extension. The cleaning of the distal end portion becomes easy, simple and effective, since the distal end portion can be designed without any undercuts and complex shapes where germs can accumulate. The coupling element is preferably designed so that it, likewise, can be easily cleaned.

The image pickup device can be part of an assembly consisting of the image pickup device and at least one lens with the lens arranged at the light incidence region at the outer circumference of the distal end portion. The distal end portion can form a housing into which the assembly consisting of the image pickup device and the at least one lens is installed such that the light incidence axis of the image pickup device points towards the lens at the exterior of the housing. Optionally, a (or several) light emitting means can be part of the said assembly. The said assembly can be designed such to be removable from the housing. The said assembly can be designed very compactly, i.e. having a small construction size. As the light incidence axis of the image pickup device points towards the exterior of the housing and is, therefore, aligned approximately radially the said assembly is short and compact in particular in the longitudinal axis direction of the distal end portion. Thus, space can be reduced even better in the longitudinal axis direction of the distal end portion.

In the housing, a blind hole with a thread can be located, which extends in the radial direction of the distal end portion, wherein a connection (e.g. provided in a circuit board or provided as a connection element to a circuit board) for the image pickup device can be provided at the bottom of the blind hole, the image pickup device can be provided adjacent to the bottom of the blind hole, and the at least one lens can comprise a male thread and can be screwed into the blind hole such that the lens is arranged in the radial direction of the image pickup device. Also with this further development, space can be reduced very well in the longitudinal axis direction from the distal end portion. By the radial alignment of the blind hole thread formed as a female thread the space-saving radial alignment of the image pickup device and the lens is predefined. This further development including the blind hole and the thread can be optionally combined with the further development including the assembly consisting of the image pickup device and the at least one lens (and, optionally, the light emitting means) such that the blind hole is formed in an assembly holder element. Alternatively, the blind hole can also be formed directly in the housing of the distal end portion.

The image pickup device can have an image pickup side, which is oriented towards the radial direction of the distal end portion. Thus, in the distal end portion, a location space provided in a shape that is adjusted to the shape of the image pickup device can be formed. Hence, the image pickup device can be arranged in the distal end portion such that a radial alignment of the image pickup device is logically obtained.

The image pickup device can be a CMOS sensor.

The endoscope can be a duodenoscope. Particularly with the duodenoscope, in the past, a prism for the transmission of light signals from a lens provided on the outside of the distal end portion to the image pickup device was used. This prism can be omitted. The light signal transmission from the lens provided on the outside of the distal end portion is performed directly to the image pickup device. In this case, the lens is arranged on the extended axis of the image pickup device.

The aspects of the present invention explained above can be combined in a suitable way.

### SHORT DESCRIPTION OF THE DRAWING

FIG. 1, in a first embodiment, discloses a schematic top view of the distal end portion of an endoscope, where the invention is applicable wherein a cap has been placed on the distal end portion.

FIG. 2 discloses a schematic top view of the distal end portion according to FIG. 1, where the cap is shown separately.

FIG. 3 discloses a side view represented as a sectional view of the distal end portion according to FIG. 2.

FIG. 4 discloses a further side view of the distal end portion according to FIG. 2.

FIG. 5 discloses a side view of a distal end portion according to a second embodiment.

FIG. 6 discloses a partial section of the side view according to FIG. 5 without an inserted optical assembly.

FIG. 7 discloses a partial section of the side view according to FIG. 5 with an inserted optical assembly.

FIG. 8 discloses a side view of the distal end portion according to FIG. 5 from the opposite side without an Albarran lever driving mechanism.

FIG. 9 discloses a side view of the distal end portion according to FIG. 5 from the opposite side with an inserted Albarran lever driving mechanism.

FIG. 10 discloses a top view of the distal end portion according to FIG. 5.

The FIGS. 11A and 11B disclose a modified arrangement of an image pickup device.

In the following, the present invention will be described in detail with reference to the drawings by referring to embodiments.

### First Embodiment

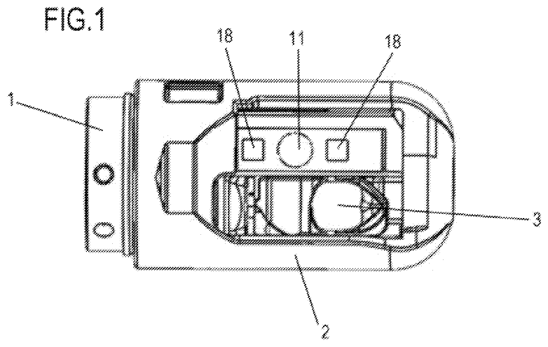

In the following, a first embodiment of the present invention will be described in general with reference to the FIGS. 1 to 4.

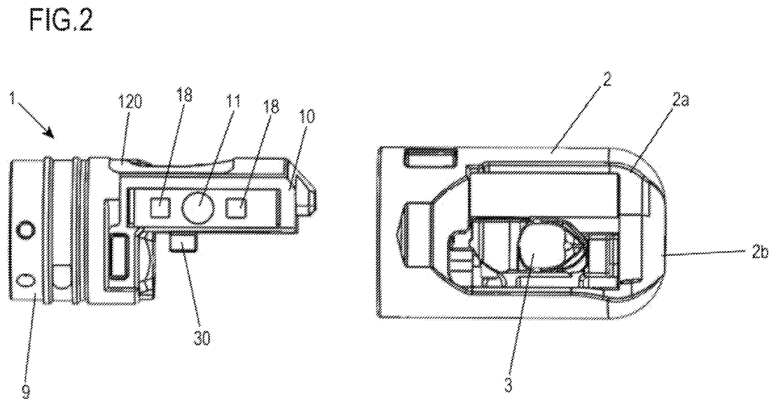

FIG. 1 discloses a schematic top view of a distal end portion 1 of an endoscope, where the invention is applicable, wherein a cap 2 is placed on the distal end portion 1. FIG. 2 discloses a schematic top view of the distal end portion 1 according to FIG. 1 with the cap being removed.

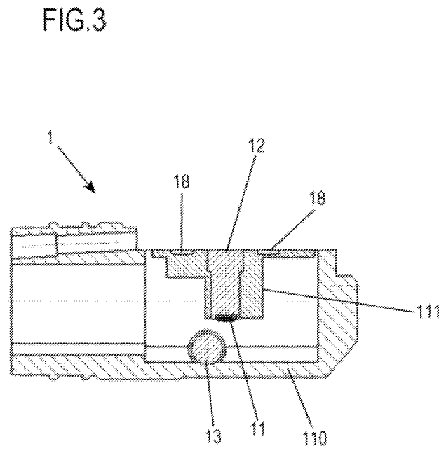

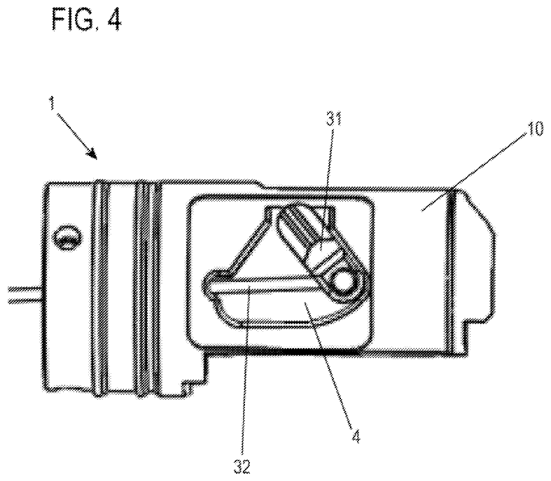

FIG. 3 discloses a side view represented as a sectional view of the distal end portion according to FIG. 2, wherein this side view is obtained when the viewer looks at the distal end portion 1 from the bottom side according to FIG. 2. FIG. 4 discloses a side view of the distal end portion from the other side according to FIG. 2, wherein this side view is obtained if the viewer looks at the distal end portion 1 from the upper side according to FIG. 2.

The distal end portion 1 can be regarded as a cylinder whose central axis extends in a manner parallel and congruent to the axis of an insertion tube of the endoscope, thus, lengthening it. The distal end portion 1 forms an endoscope head that follows the insertion tube in the distal direction.

On the distal end portion 1, a protecting cap 2 is placed from the distal side. The cap 2 is removable from the distal end portion 1 by pulling it from the distal end portion 1 in the distal direction. The cap 2 is preferably designed as a single-use body. The cap 2 forms a cup having a bottom part 2b. The bottom part 2b of the cap 2 is shown in FIG. 2 on the right side of the cap 2.

The cap 2 has a window 2a. The window 2a is an opening in the circumferential wall of the cap 2. The window 2a provides access to the interior of the cap 2. On an inner circumferential wall of the cap 2, an Albarran lever (elevator) 3 is pivoted. Therefore, a bearing is provided on the inner circumferential wall of the cap 2. The said bearing forms a pivot of the Albarran lever 3. A proximal portion of the Albarran lever 3 is pivoted on the said bearing. The Albarran lever 3 is, thus, movably integrated in the cap 2. Hence, whenever the cap 2 is removed from the distal end portion 1 the Albarran lever is likewise removed.

The window 2a therefore provides access to the Albarran lever 3, to an image pickup device 11 and to a light emitting means 18 described in the following.

The distal end portion 1 is designed as a housing 110. The distal end portion 1 has a main body 9 on the proximal side and a distal extension 10. On the proximal side, the main body 9 is connected to the not shown insertion tube of the endoscope. On the proximal side of the insertion tube, a control unit of the endoscope is provided.

The main body 9 has a cylindrical shape. The distal extension 10 extends from the main body 9 in the distal direction. In this case, the distal extension 10 only extends from a section of the distal side of the main body 9. Another section of the distal side of the main body 9 forms a free space. In FIG. 2, the free space is shown below the distal extension 10. In the said free space, the Albarran lever 3 can be arranged.

The main body 9 and the distal extension 10 are formed integrally and, together, they represent the housing 110.

The image pickup device 11 and the light emitting means 18 are arranged in the distal extension 10. FIG. 3 shows the more detailed design in a sectional view. On a side of the distal extension 10 shown at the upper side of FIG. 3, in a wall of the housing 110, a distal LED 18 and a proximal LED 18 are provided as a light emitting means, which are operated by the control unit of the endoscope. The invention is not limited to two LEDs 18. The number of LEDs 18 can be three or more. Alternatively, also only one LED 18 can be used.

In view of FIG. 3, in the wall of the housing 110, a perpendicularly extending blind hole 111 is provided in the area between the distal LED 18 and the proximal LED 18. The blind hole 111 extends in the radial direction of the distal extension 10. The blind hole 111 has a bottom and a circumferential wall. The image pickup device 11 is located at the bottom of the blind hole 111. The image pickup device 11 can be provided as a CMOS sensor. The image pickup device 11 has an axis, which is, in its installation position, radially aligned. In the example of FIG. 4, the image pickup device 11 is a flat cylindrical body, which is placed at the bottom of the blind hole 111. The image pickup device 11 receives image data of the surrounding area and transmits them for evaluation to the control unit of the endoscope.

In view of FIG. 3, a lens 12 is located in the blind hole 111 above the image pickup device 11 (i.e. radially from the image pickup device 11). According to the first embodiment, the lens 12 is glued into the blind hole 111. The lens 12 transmits the light signals received on the light incidence side to the image pickup device 11. The lens 12 is designed as a longitudinal cylindrical body. The axis of the lens 12 extends in the radial direction. The lens 12 forms a light incidence region at the outer circumference of the distal end portion 1.

The overall design of the image pickup device 11 and the light emitting means 18 is very compact as a result of the radial alignment of blind hole 111, image pickup device 11 and lens 12, and takes up very little space in the extension direction (longitudinal direction, axial direction) of the distal extension 10. Thus, space can be saved in the distal extension 10, which is usable for other purposes.

Furthermore, in the distal extension 10, a chamber 4 for accommodating the Albarran lever driving mechanism is provided. So far, in many designs of endoscopes, an Albarran lever driving mechanism could not be located in a distal extension such as the distal extension 10, since the equipment for the image pickup device and the light emitting means required a lot of space in the distal extension. Now, the said chamber 4 is integrated likewise in the distal extension 10 according to the invention.

FIG. 4 shows the distal extension 10 with regard to the upper side according to FIG. 2. FIG. 4 shows the design of the chamber 4. The chamber 4 is a recess in the housing 110 provided in the distal extension 10. The chamber 4 is open only towards the side which, according to FIG. 4, points to the viewer and, in the proximal direction, through a pulling cable channel. The side according to FIG. 4 pointing to the viewer represents an access side to the chamber 4. The said access side is tightly closed by a cover, which is not shown in FIG. 4. The cover can be removed from the housing of the distal extension 10. The pulling cable channel represents the only unsealed access to the chamber 4.

The chamber 4 comprises a bearing for a shaft 30 of the Albarran lever 3. The bearing for the shaft 30 extends from the chamber 4, through the housing 110, to the side of the free space. On the side of the free space, an end of the shaft 30 opposed to the chamber 4 protrudes, as shown in the FIGS. 2 and 3.

An end of the drive lever 31 of the Albarran lever 3 is located at the end of the shaft 4 located in the chamber 4. The drive lever 31 has a reception part on the side opposed to the end of the shaft 4, into which a distal nipple of a pulling cable 32 is inserted. The pulling cable 32 extends from the drive lever 31, through the pulling cable channel, to the control unit of the endoscope and is pulled or relieved in a manner known to the skilled person by a lever or an actuator. By pulling the pulling cable 32, the drive lever 31 and, thus, also the shaft 4 and the Albarran lever 3 placed on the shaft 4 on the side of the shaft 4 opposed to the drive lever 31 are rotated.

Hence, only the drive lever 31, the distal end of the pulling cable 32 and the end of the shaft 4 protruding into the chamber 4 are located in the chamber 4. The chamber 4 is sealed towards the outside. Germs cannot penetrate into the chamber 4.

### Second Embodiment

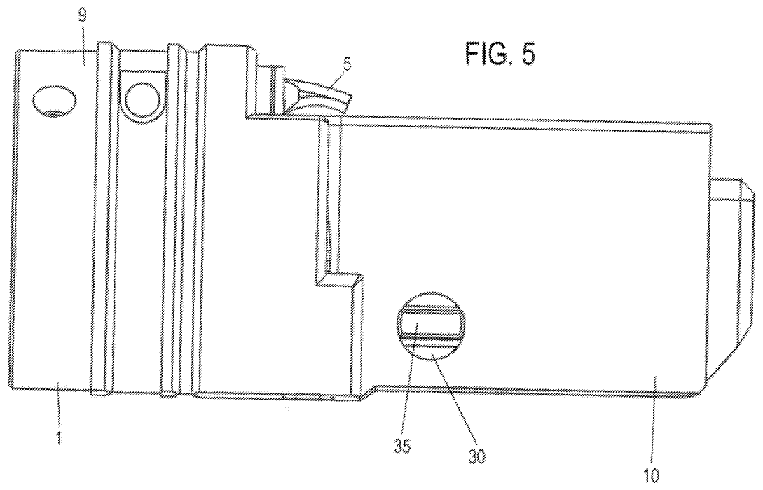

In the following, a second embodiment of the present invention is described in detail with reference to the FIGS. 5 to 10.

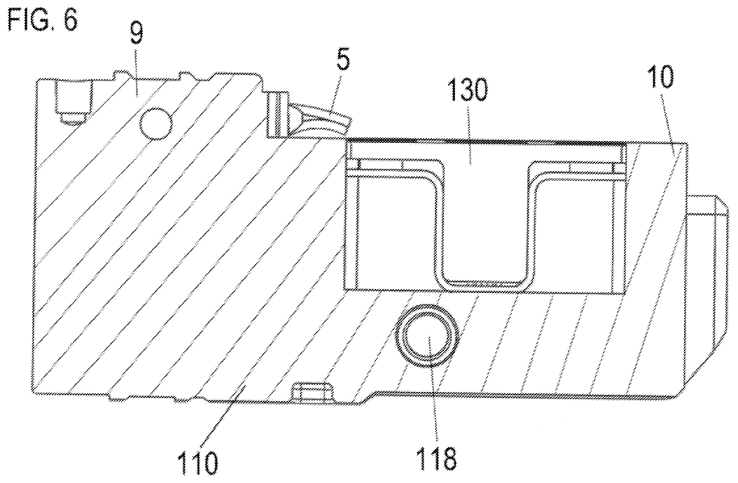

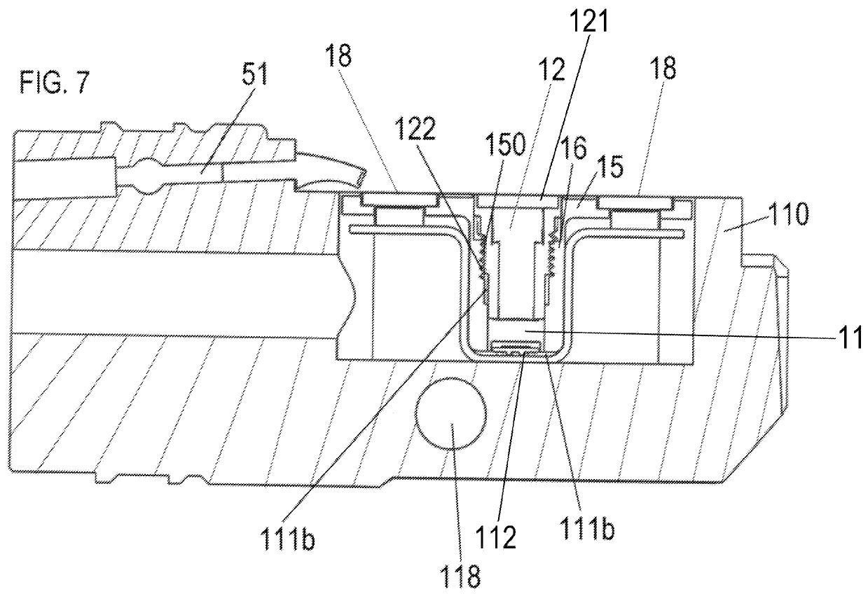

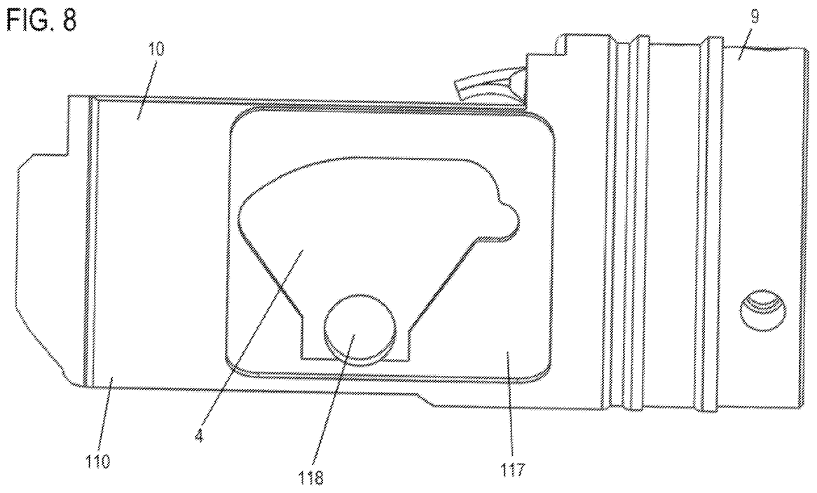

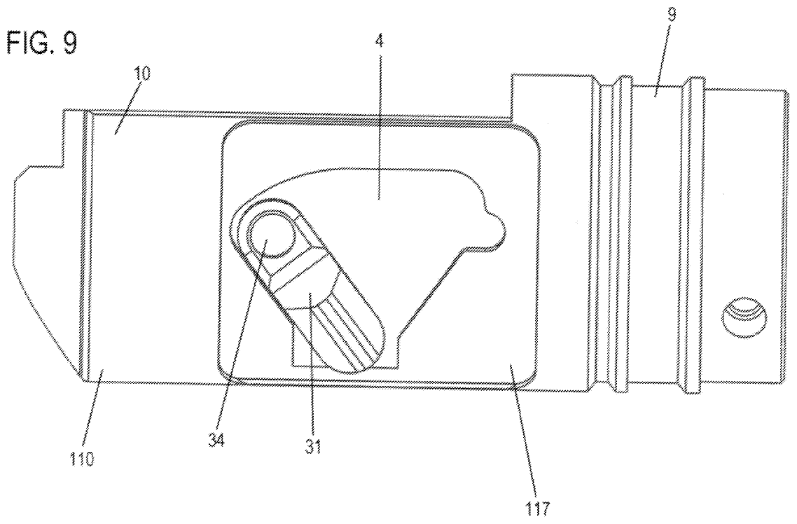

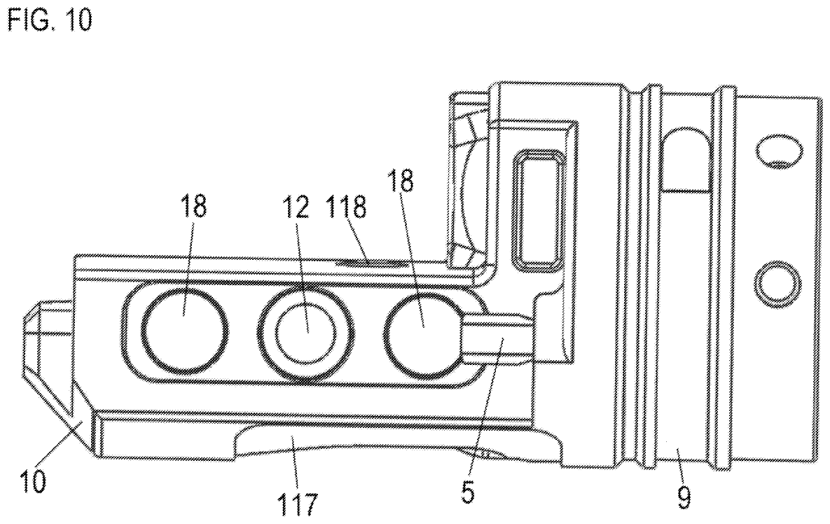

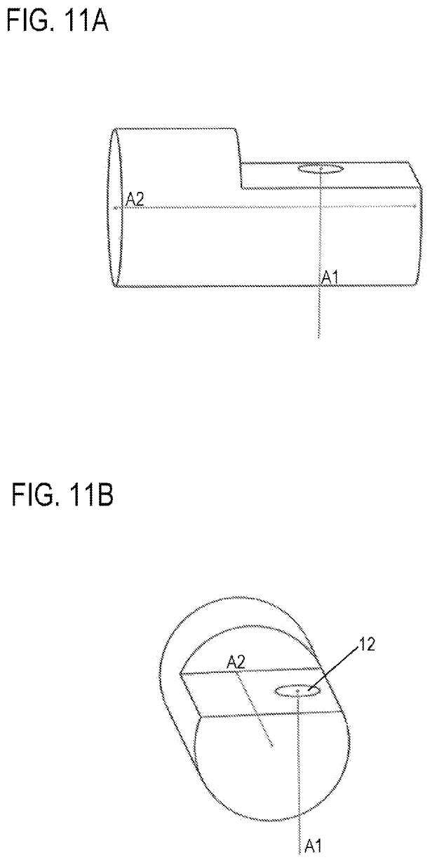

FIG. 5 shows a side view of the distal end portion 1 according to the second embodiment from a side, on which a free space is obtained like in the first embodiment. The FIGS. 6 and 7 show the distal end portion 1 from the same side. The FIGS. 8 and 9 show the distal end portion 1 from the opposite side. FIG. 10 shows a top view of the distal end portion 1. Like in the first embodiment, the distal end portion 1 has a cylindrical proximal main body 9, from which a distal extension 10 extends in the distal direction.

With reference to FIG. 10, the one side with the free space above the distal extension 10 is shown. Thus, the FIGS. 5 to 7 show the distal end portion 1 from the upper side in FIG. 10. The FIGS. 8 and 9 show the distal end portion 1 from the bottom side in FIG. 10.

In the second embodiment, the distal end portion 1 has a flushing nozzle 5, which can dispense a cleaning liquid onto an outer surface of a LED 18 and an outer surface of a lens 12 described below, see FIGS. 5 and 10. The flushing nozzle 5 is located at the upper side of the distal extension 10. A flushing channel 51 extends through the main body 9. The flushing channel 51 is, on the proximal side, fluid-connected to a liquid source. The flushing channel 51 supplies the cleaning liquid of the liquid source to the flushing nozzle 5.

Like in the first embodiment, a housing 110 is formed integrally of the main body 9 and the distal extension 10.

A recess 130 is located at the upper side of the housing 110 in the distal extension 10. The recess 130 forms an opening accessible from the upper side, see FIG. 6. An optical assembly 15 as described below will be located in the recess 130. The recess 130 is adjusted to the shape of the optical assembly 15. If the optical assembly 15 is inserted into the recess 130, see FIG. 7, the recess is closed and sealed. The optical assembly 15 can be glued into the recess 130.

The optical assembly 15 is formed integrally of a carrier 16 and a LED 18 as light emitting means. In the carrier 16, the LED 18 is integrally integrated but can also be fastened by gluing or otherwise. In the present example, two LEDs 18 are shown. However, the number of LEDs 18 is not limited. The carrier 16 comprises a blind hole 111. In the installation position of the carrier 16 in the distal extension 10, the blind hole 111 is located in the radial direction relative to the distal extension 10. Functionally, the blind hole 111 corresponds to the blind hole 111 of the first embodiment.

The blind hole 111 has a female thread 150 provided on an inner circumferential wall 111b of the blind hole. Furthermore, the blind hole 111 has a bottom 111a. A not shown circuit board can be provided in the housing 110 in the blind hole bottom 111a, at the blind hole bottom 111a, in the area below the blind hole bottom 111a or spaced from the blind hole bottom 111a. The said circuit board serves as the electrical supply of e.g. the LED 18 and a camera body 11 as image pickup device.

The camera body 11 can be a CMOS sensor. The camera body 11 is located at the bottom 111a of the blind hole. The image pickup side of the camera body 11 points towards the radial direction of the distal end portion 1. In FIG. 7, the image pickup side of the camera body 11 points upwards. In other words, the image pickup side of the camera body 11 points to the extension direction of the blind hole 111. A connection 112 (e.g. of a circuit board) is in contact with the camera body (image pickup device) 11 to transmit signals. The said signals can be control signals controlling the image pickup device 11, and image transmission signals that are sent from the pickup device 11 to the control unit of the endoscope.

A lens 12 is screwed into the blind hole 111 above the camera body 11 (in the radial direction from the camera body 11). The lens 12 has a male thread 122. The male thread 122 of the lens 12 is in thread engagement with the female thread 150 of the blind hole 111. The lens 12 is an optical system, which serves for light transmission to the image pickup device 11 and includes suitable optical elements. One of these optical elements is a lens 121 provided on the light incidence side of the lens 12.

Thus, the camera body 11 is located in the distal end portion 1 such that an image information falls through the lens 12 in the radial direction directly onto the image pickup side of the camera body 11. Consequently, the image pickup side of the camera body 11 is parallel to a light incidence surface on the radial outside of the lens 12 and it is located on the same axis, see FIG. 7.

On the side opposite of the free space, a chamber 4 for accommodating an Albarran lever driving mechanism is located in the distal extension 10.

The Albarran lever driving mechanism consists of a drive lever 31 of a not shown Albarran lever. Like in the first embodiment, the drive lever 31 is connected to a shaft 30, which is inserted into the Albarran lever if a not shown cap is placed on the distal end portion 1. Also in the second embodiment, the Albarran lever is pivoted in the cap. In the present embodiment, an Albarran lever and a cap can be used like the Albarran lever 3 and the cap 2 of the first embodiment.

The end of the shaft 31 pointing to the side of the free space has a connection protrusion 35 for the Albarran lever. The connection protrusion 35 forms a coupling element for the Albarran lever. The Albarran lever 3 is supported in a hinged manner, i.e. pivoted, on the distal extension 10 through the connection protrusion 35. The connection protrusion 35 protrudes to the side of the free space and it is designed as a flat, cuboid protrusion. As shown in FIG. 5, in idle position of the Albarran lever driving mechanism, the upper and lower flat surface of the connection protrusion 35 extend in the direction that corresponds to the longitudinal direction of the distal extension 10. Same corresponds to the pushing direction of the cap. The Albarran lever has a recess, which corresponds, in view of the shape, to the connection protrusion 35. Thus, when pushing the cap onto the distal end portion 1, the Albarran lever is pushed on the connection protrusion 35 and reaches a positive connection with the connection protrusion 35. Thereby, the Albarran lever is pivotable by the Albarran lever driving mechanism if the cap is pushed onto the distal end potion 1.

An opening 118 representing a through hole extends from the chamber 4 to the side of the free space through the distal extension 10, i.e. through the housing 110. The opening 118 is located in the housing 110 as a bearing for the shaft 30 of the Albarran lever 3. The shaft 30 is rotatably mounted in the opening 118. Between the shaft 30 and the opening 118, a not shown sealing is provided. Thus, the chamber 4 is sealed towards the side of the free space.

From the proximal side, a not shown pulling cable channel leads to the chamber 4. Like in the first embodiment, a pulling cable passes through the pulling cable channel. At one end of the drive lever 31, the pulling cable is hooked into an opening 34 via a nipple of the pulling cable. The other end of the drive lever 31 is connected to the shaft 30.

On the side opposite to the side of the free space, the distal extension 10 has a recess 117 enclosing the chamber 4 for covering the chamber 4. Like in the first embodiment, a not shown cover (a cap) is put tightly on the recess 117 and e.g. fixed by gluing.

Thus, the chamber 4 is sealed towards the side opposite of the side of the free space.

### Effects of the Present Invention

The following advantageous effects are achieved by the invention, which will be explained by referring to exemplary embodiments.

(1) The camera (i.e. the image pickup device) is located radially to the distal end. The image pickup surface of the camera points to the radial direction. The image pickup surface of the camera and the image pickup surface of the lens are located on a radial line. The image pickup surface of the camera and the image pickup surface of the lens are not offset in the longitudinal direction of the endoscope.

This design minimizes the space required for the camera and the lens in the longitudinal direction of the endoscope. In the longitudinal direction of the endoscope, the space distal and/or proximal to the camera and the lens can be used for a different purpose.

(2) The camera, the lens, a circuit board including electronic components for the camera and the illumination (LED) and, in addition, the complete control mechanism of the Albarran lever are located on one side of the distal end.

(3) The other side of the distal end becomes a free space. This free space is easy to clean. Only the connection protrusion 35 of the shaft 30 for the Albarran lever slightly protrudes into the free space. The connection protrusion 35 is cuboid-shaped, having surfaces extending in the longitudinal direction of the endoscope. This design optimizes the cleaning capability of the endoscope at its distal end.

(4) The endoscope according to the invention has the removable assembly consisting of cap and Albarran lever. For treatment, the cap with the Albarran lever are removed and can be disposed of. The one side of the distal end forms the free space. Thus, it is completely open and easy to clean.

(5) In the present invention, the camera is aligned radially to the distal end and, therefore, space for the control mechanism of the Albarran lever is created. The control mechanism is shifted to the side where the camera is located. The other side of the distal end is free of components and, thus, easier to treat.

The cap with the Albarran lever is used once. After every examination it has to be disposed of. The one open side of the distal end forms a free space and improves the cleaning capability of the distal end. This is possible since the camera and the control mechanism of the Albarran lever are located on the same side of the distal end.

### Alternatives

According to the first and second embodiment, the image pickup device (camera) 11 is installed in the housing 110 of the distal end portion such that the longitudinal axis is aligned perpendicularly to the outer surface of the distal extension 10, at which the lens 12 and the LED 18 are located. This perpendicular alignment means a 90 degree angle, as disclosed by the drawings of the first and second embodiment. The invention is not limited to this design. The angle can also be 85 to 95 degrees. In a further alternative, the said angle can also be 75 to 105 degrees. In again another alternative, the said angle can be 70 to 110 degrees. Also with the said angles differing from 90 degrees, the effect of the invention is achieved. Also with the said angles, the image pickup device (camera) 11 can be installed in the housing 110 of the distal end portion such that the longitudinal axis of the image pickup device (camera) 11 extends perpendicularly to the lens 12 on the outer surface of the distal extension 10. Therefore, also with these alternatives, a prism between the camera and the lens is not necessary.

The FIGS. 11A and 11B show such a modified arrangement of an image pickup device. FIG. 11A shows a side view of longitudinal extension of the distal end portion. FIG. 11B shows a perspective schematic view from the distal side. A1 refers to the longitudinal axis of the image pickup device (camera). A2 refers to the longitudinal axis of the distal end portion. As disclosed by FIG. 11B, the longitudinal axis A1 of the image pickup device (camera) can be arranged at an angle differing from an exact vertical angle with regard to the outer surface of the distal extension.

According to the first embodiment, a distal LED 18 and a proximal LED 18 are provided as light emitting means. The invention is not limited to this design. Apart from LEDs, other light emitting means can be used. E.g. a suitable number of optical fibers can be used.

According to the first embodiment, the lens 12 is installed in the housing 110, e.g. glued in. According to the second embodiment, the optical assembly 15 is placed in the housing 110, e.g. glued in. The optical assembly 15 has the blind hole 111 including a thread, into which the lens 12 is screwed. The invention is not limited to this design. Alternatively, a blind hole with a thread can also be directly provided in the housing 110. According to this variant, the lens 12 can be directly screwed into the housing 110.

The method of fastening the lens 12 and the image pickup device 11 is not restricted to a gluing or screwing into the housing 110. The lens 12 and image pickup device 11 can be fixed in the housing 110 in whatever way as long as a radial alignment of the image pickup device 11 is ensured.

According to the second embodiment, the flushing nozzle 5 is not necessary.

The invention can be advantageous for a duodenoscope, a gastroscope, a colonoscope or any similar endoscope. The principle of the invention can also be applied to any other kind of endoscope.

### LIST OF REFERENCE SIGNS

1 distal end portion

2 cap

2a cap window

2b cap bottom

3 Albarran lever

4 chamber for accommodating the Albarran lever driving mechanism

5 flushing nozzle

9 main body of the distal end portion

10 distal extension

11 image pickup device

12 lens, light incidence region

15 optical assembly

16 carrier

18 light emitting means (LED)

30 Albarran lever shaft

31 drive lever of the Albarran lever

32 pulling cable

33 pulling cable channel

34 opening for the nipple of the pulling cable

35 connection protrusion of the shaft for the Albarran lever

51 flushing channel

110 housing

111 blind hole

111a bottom of the blind hole

111b circumferential wall

112 connection for the image pickup device

117 recess in the housing for the chamber cover

118 opening in the housing for the Albarran lever shaft

120 cover of the chamber 4

121 lens

122 male thread of the lens

130 recess

150 female thread

1. An endoscope having a distal end portion at which a light emitter and an image pickup device are arranged, wherein, at the distal end portion, a light incidence region is defined at the outer circumference of the distal end portion,
characterized in that
the longitudinal axis of the image pickup device is oriented towards the light incidence region at the outer circumference of the distal end portion.

2. The endoscope according to claim 1, wherein the longitudinal axis of the image pickup device extends perpendicularly to the light incidence region at the outer circumference of the distal end portion.

3. The endoscope according to claim 1, wherein
the distal end portion comprises a distal extension extending from the main body of the distal end portion in a distal direction;
the distal end portion comprises an Albarran lever extending from the main body of the distal end portion in a distal direction; and
the image pickup device is inserted at the distal extension.

4. The endoscope according to claim 3, wherein
the Albarran lever is pivotally supported on the distal extension.

5. The endoscope according to claim 1, wherein
the image pickup device forms part of an assembly comprised of the image pickup device and at least one lens, with the lens being arranged at the light incidence region at the outer circumference of the distal end portion; and
the distal end portion forms a housing into which the assembly comprised of the image pickup device and the at least one lens are installed such that the light incidence axis of the image pickup device points towards the lens at the exterior of the housing.

6. The endoscope according to claim 5, wherein
in the housing, a blind hole with a thread is provided which extends in the radial direction of the distal end portion;
a connection for the image pickup device is provided at the bottom of the blind hole;
the image pickup device is arranged adjacent to the bottom of the blind hole; and
the at least one lens comprises a male thread and is screwed into the blind hole such that the lens is arranged in the radial direction of the image pickup device.

7. The endoscope according to claim 1, wherein
the image pickup device has an image pickup side which is oriented towards the radial direction of the distal end portion.

8. The endoscope according to claim 1, wherein
the image pickup device is a CMOS sensor.

9. The endoscope according to claim 1, wherein
the endoscope further comprises
an optical assembly that is formed of a carrier and the light emitter as an integral assembly, the optical assembly being arranged in a recess of the housing,
in the carrier a blind hole is provided, the blind hole having a female thread, and
the lens has a male thread, the lens being screwed into the carrier by the male thread being in thread engagement with the female thread.

10. The endoscope according to a claim 1, wherein
the endoscope is a duodenoscope.
